(19) United States
(12) Patent Application Publication
(10) Pub. No.: US 2021/0353672 A1
(43) Pub. Date: Nov. 18, 2021

(54) COMPOSITIONS FOR ADMINISTRATION TO RUMINANT ANIMALS

(71) Applicant: D.I.T. TECHNOLOGIES LTD., Toowoomba, Queensland (AU)

(72) Inventors: Thomas Robinson Feez, Withcott (AU); Mark Rawlinson Peart, Geham (AU); Anthony David Wood, Corinda (AU)

(21) Appl. No.: 17/287,105

(22) PCT Filed: Oct. 3, 2019
(86) PCT No.: PCT/AU2019/051072

§ 371 Date: Apr. 20, 2021

(30) Foreign Application Priority Data
Oct. 23, 2018 (AU) 2018904024

(51) Int. Cl. A61K 33/34; A61K 33/06; A61K 33/30; A61K 33/04; A61K 33/32; A61K 33/18; A61K 33/24; A61K 31/7004; A61K 9/00; A61P 25/22

(52) U.S. Cl. CPC A61K 33/34; A61K 33/06; A61K 33/30; A61K 33/04; A61K 33/32; A61P 25/22; A61K 33/24; A61K 31/7004; A61K 9/0095; A61K 9/0053; A61K 33/18

(57) ABSTRACT

The present invention relates to a composition for administration to ruminant animals in their drinking water to reduce transport stress. The composition comprises a carbohydrate capable of acting as an energy source and/or carbon source for amylose degrading bacteria such as dextrose, a source of magnesium and selected trace elements. The composition is effective in stimulating cellulose digestion in the rumen, in an increasing net available energy and reducing physiological stress. In turn, this leads to greater disease resistance and calmer animals as well as a greater utilisation of the available diet, or creates an energy sparing effect for existing diets.

### TECHNICAL FIELD

The present invention relates to compositions for administration to ruminant animals. More particularly, the present invention relates to compositions for administration to ruminant animals in drinking water.

### BACKGROUND

Any references to methods, apparatus or documents of the prior art are not to be taken as constituting any evidence or admission that they formed, or form part of the common general knowledge.

Cattle and sheep deaths during the transport, handling and yarding process are still common and largely preventable. Transport tetany, also known as transit tetany, railroad disease, railroad sickness, or staggers is a disease that occurs in cows and ewes after the stress of prolonged transport. Lactating cows and young cattle or cattle in a weakened condition are most susceptible. These animals may die a few days after arrival, or in severe cases, will die on the trucks or within hours of arrival

Other problems associated with transport stress include lesser resistance to bruising and hide damage as well as transport shrink in weight. Cattle that have been subjected to undue stress before slaughter have reduced amounts of muscle glycogen. When the animal is slaughtered, the muscle glycogen is converted to lactic acid which causes the pH to fall. Animals with reduced glycogen levels produce less lactic acid so the meat has a relatively high pH. Such animals are referred to as “dark cutters” since beef cut from these animals has a dark colour which makes the meat appear less fresh, making it undesirable to consumers.

Transport stress can also cause losses in production due to decreased disease resistance, poor appetite and extended recovery periods required following stressful transport. Cattle and sheep taken off feed, handled and transported for any distance undergo a series of physiological changes that lead to muscular exhaustion, imbalance in electrolytes and metabolic changes that can take a considerable time to reverse. This is commonly seen in cattle transported to saleyards or feedlots where the transport shrink may be up to 12% and the time taken to reverse the metabolic changes and for cattle to return to normal growth patterns may be 10 days or longer. Some cattle may arrive in a completely exhausted state and may not completely recover.

There is also an adverse effect on the immune competence of the animal and this stress will cause an increased susceptibility to disease, particularly virus infections. This is apart from any physical damage to the animal due to poor yard design or inadequate trucks, which can be increased if the animal is in a poor physical state due to transport stress.

From an economic viewpoint, these factors are vitally important since they can impair meat quality and increase carcass loss in cattle as well as causing production losses in cattle introduced to varying feedlot conditions. Furthermore, there is a growing concern that animal welfare conditions in the transport and handling environment are severely degraded and that this is preventable.

The major conditions that occur during and following handling and transport are muscular exhaustion, metabolic acidosis, subclinical ketosis, dehydration, tissue catabolism, and ruminal atony, decreased levels of calcium and magnesium ions and increased susceptibility to infections due to loss of immune competence. These lead to reduced appetite, slow recovery and increased susceptibility to infections.

### SUMMARY OF INVENTION

The present invention is based on the observation that administration of a carbohydrate capable of acting as an energy source and/or carbon source for amylose degrading bacteria in conjunction with magnesium and selected trace elements in drinking water is effective in stimulating cellulose digestion in the rumen, in increasing net available energy and reducing physiological stress. In turn, this leads to greater disease resistance and calmer animals as well as a greater utilisation of the available diet, or creates an energy sparing effect for existing diets.

In one aspect there is provided a composition for reducing transport stress in a ruminant animal which is suitable for administration in the drinking water of the ruminant animal, comprising:
(a) a carbohydrate capable of acting as an energy source and/or carbon source for amylose degrading bacteria;
(b) a source of magnesium; and
(c) trace elements comprising:
   a source of iodine;
   a source of cobalt;
   a source of copper;
   a source of zinc;
   a source of selenium; and/or
   a source of manganese.

In a further aspect there is provided a method of reducing transport stress in a ruminant animal, comprising administering:
(a) a carbohydrate capable of acting as an energy source and/or carbon source for amylose degrading bacteria;
(b) a source of magnesium; and
(c) trace elements comprising:
   a source of iodine;
   a source of cobalt;
   a source of copper;
   a source of zinc;
   a source of selenium; and/or
   a source of manganese
      in the drinking water of the ruminant animal.

In a further aspect there is provided a composition comprising
(a) a carbohydrate capable of acting as an energy source and/or carbon source for amylose degrading bacteria;
(b) a source of magnesium; and
(c) trace elements comprising:
   a source of iodine;
   a source of cobalt;
   a source of copper;
   a source of zinc;
   a source of selenium; and/or
   a source of manganese;
      for use in reducing transport stress in a ruminant animal.

In a further aspect there is provided a kit comprising
(a) a carbohydrate capable of acting as an energy source and/or carbon source for amylose degrading bacteria;
(b) a source of magnesium;
(c) trace elements comprising:
   a source of iodine;
   a source of cobalt;
   a source of copper;
   a source of zinc;
   a source of selenium; and/or
   a source of manganese; and
(d) instructions for administering the carbohydrate, source of magnesium and trace elements to the drinking water of a ruminant animal.

### DETAILED DESCRIPTION OF PREFERRED EMBODIMENTS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way.

Ruminant nutrition is totally dependent on the efficiency of microbial fermentation in the rumen. Ruminants have adapted to a variety of ecological niches because they have diverse ruminal microbial populations, which consist primarily of bacteria, archaea, protozoa and fungi. Ruminant animals have the ability to convert low quality feeds into high quality protein and to utilize feeds from a variety of environments. This is made possible by the ruminal microorganisms that synthesize and secrete the β 1-4 cellulase enzyme complex, thereby allowing hydrolysis of plant cell walls. However, the actual conversion of feeds, especially fibrous forages, to meat and milk is not very efficient. Only 10-35% of energy intake is captured as net energy because 20-70% of cellulose may not be digested. If a greater percentage of the total dietary energy from forages was available to ruminants, lower cost diets could be formulated and environmental resources would be used more efficiently.

Microbial communities exist in the rumen in discrete, structured and organised communities that control the complex hydrolytic and enzymatic breakdown of feed. The microbes exist in a biofilm matrix where products used by one colony are used or required by closely associated colonies. The rumen fermentation system has evolved to convert cellulose to volatile fatty acids and high quality protein that can be utilised for growth by the host.

The microbial colonies are encased in polymeric substances that produce the biofilm and grow inward to access the fermentable materials of the plant substrates. The fungi aid this process by invading the plant particle, weakening the structure and allowing access for other organisms, as well as contributing to the breakdown of lignin and hemicellulose.

The main fermenters are the bacteria and they can establish biofilm colonies on new ingesta in less than 1 hour. They can also communicate with other colonies and rapidly respond to changing conditions. There are also complex and synergistic relationships between the motile protozoa and the bacteria, the protozoa helping the bacteria reach the site of their substrate by physically carrying them to the site as well as sharing and utilising each other's metabolic products.

Enzymes are the products of microbes that bring about the degradation of the polymer substrate that are present in the plant biomass. The bovine rumen is an anaerobic or microaerophilic environment where the microflora are a rich source of the enzyme groups required to complete digestion. The fibrolytic enzymes are needed to degrade components of plant cell walls (such as hemicellulases, xylases, arabinofuranosidases, cellulases, glucanohydrolases, glucosidases, and endoglucanases), while laccases (phenol oxidases) and peroxidases (lignin peroxidases) are important plant polymer modifying enzymes that facilitate digestion of lignin. The discovery of oxidative enzymes, laccases and peroxidases in the rumen indicate that these lignin-breaking enzymes are also important in rumen digestion.

Trace elements such as copper, manganese and zinc are important in that many enzymes require the presence of these compounds in their molecular structure. For example, manganese peroxidase is an important oxidative enzyme and some laccases have four copper molecules at their centre. Other trace minerals such as cobalt and selenium are important in that microbial fermentation converts these to B group vitamins and glutathione peroxidase respectively. While not wishing to be bound by theory, it is believed that the provision of the trace elements in the formula ensures that these important trace minerals are readily available to the biofilm in which microbial fermentation reactions are occurring.

While rumen fermentation is primary to digestion, physiological factors that affect the animal are also important in ensuring growth. Animals that are severely stressed or agitated may have higher body temperatures, higher base metabolism and lower appetite, do not ruminate (cud chewing) as normal and will not gain weight as well as those that are not stressed. In addition, stress may reduce immune function and disease resistance. It is believed that the administration of a composition comprising a carbohydrate, a source of magnesium and trace elements positively influences these physiological factors.

The composition is adapted for administration to ruminant animals in drinking water. Selection of sources of trace elements and/or carbohydrates which are water soluble is required. As used herein the term “water soluble” or references to water solubility means that a chemical compound is capable of dissolving in water or a material that contains the element in question is capable of dissolving in water, more or less completely. In order to dissolve more or less completely there will be little or no solid residue in the water after a reasonable time has elapsed and where reasonable mixing steps have been undertaken. Optionally, there can be addition of surfactants or other additives that ensure miscibility with drinking water. The composition may be a dry mix that is soluble in water. The composition may also be presented in liquid form. Typically, this will be in the form of an aqueous solution.

A physiologically acceptable composition will usually comprise at least one adjuvant, diluent or carrier, which may be selected with due regard to the intended route of administration and standard practice in formulating supplements. Such carriers may be chemically inert to the active compounds and may have no detrimental side effects or toxicity under the conditions of use. The preparation of suitable formulations may be achieved routinely by the skilled person using routine techniques and/or in accordance with standard and/or accepted pharmaceutical practice.

In an embodiment, the source of iodine is a compound selected from the group consisting of lithium iodide, sodium iodide, potassium iodide, ammonium iodide, magnesium iodide, calcium iodide, zinc iodide and iron iodide.

In an embodiment, the source of iodine is substantially 0.01 to 0.50% of total agent composition. More preferably, source of iodine is substantially 0.06 to 0.31% of total agent composition.

In an embodiment, the source of cobalt is a compound selected from the group consisting of cobalt chloride, cobalt chlorate, cobalt bromide, cobalt bromate, cobalt iodide, cobalt iodate, cobalt nitrate and cobalt sulfate.

In an embodiment, the source of cobalt is substantially 0.01 to 1.00% of total agent composition. More preferably, source of cobalt is substantially 0.02 to 0.92% of total agent composition.

In an embodiment, the source of copper is a compound selected from the group consisting of copper bromide, copper chloride, copper chlorate, copper selenate and copper sulfate.

Preferably, the source of copper is substantially 0.01 to 1.00% of total agent composition. More preferably, source of cobalt is substantially 0.25 to 0.70% of total agent composition.

In an embodiment, the source of zinc is a compound selected from the group consisting of zinc acetate, zinc bromide, zinc chlorate, zinc chloride, zinc iodide, zinc nitrate and zinc sulfate.

In an embodiment, the source of zinc is substantially 0.50 to 2.00% of total agent composition. More preferably, source of zinc is substantially 0.81 to 1.93% of total agent composition.

In an embodiment, the source of selenium is a compound selected from the group consisting of ammonium selenate, calcium selenate, copper selenate, magnesium selenate and potassium selenate.

In an embodiment, the source of selenium is substantially 0.01 to 0.50% of total agent composition. More preferably, the source of cobalt is substantially 0.01 to 0.20% of total agent composition.

In an embodiment, the source of manganese is a compound selected from the group consisting of manganese bromide, manganese chloride, manganese nitrate and manganese sulfate.

In an embodiment, the source of manganese is substantially 0.50 to 2.50% of total agent composition. More preferably, source of cobalt is substantially 0.85 to 2.01% of total agent composition.

In an embodiment, the physiologically acceptable composition also comprises a source of magnesium. Magnesium exerts a calming effect. For example, magnesium fed to animals before slaughter tempers the action of stress on muscle glycogen by blocking the effect of adrenaline.

In an embodiment, the source of magnesium is a compound selected from the group consisting of magnesium acetate, magnesium bromide, magnesium bromate, magnesium chloride, magnesium chlorate, magnesium chromate, magnesium iodide, magnesium iodate, magnesium molybdate, magnesium nitrate, magnesium selenate and magnesium sulfate.

In an embodiment, the source of magnesium is substantially 0.1 to 10% of total agent composition. In an embodiment, the source of magnesium is substantially 0.2 to 5% of total agent composition. In an embodiment, the source of magnesium is substantially 0.20 to 1.50% of total agent composition. In an embodiment, the source of magnesium is substantially 0.36 to 1.00% of total agent composition.

While not wishing to be bound by theory, it is believed that the integrated physiological effects of calming, greater relaxation and increased rumination lead to positive effects on digestion. Accordingly, administration of a carbohydrate, a source of magnesium and trace elements to ruminant animals leads to greater activity of the ruminal microorganisms and produces the outcome of increased cellulose digestion.

Livestock animals such as cattle and sheep are herbivores, and so derive much of their energy requirements from cellulose. The digestion of cellulose in the rumen requires the interaction of both cellulolytic and non-cellulolytic bacteria, as well as protozoa. Major cellulolytic species include: Fuminococcus albus, Ruminococcus flavzfaciens, Bacteroides succinogenes, and Butyrivibrio fibrisolvens. Of these, Bacteroides succinogenes is the most active in digestion of cellulose, especially the more resistant forms. These organisms form a biofilm on particles of plant material that enter the rumen. A feature of the biofilm that covers the plant particle is that the metabolic end products of one species is a substrate for a nearby species. Syntrophism exists between species—this being an interaction that occurs when metabolically different bacteria depend on each other to be able to degrade particular substrates and share the energy released for their maintenance and growth. For example, the amylose degrading bacteria convert sugars to isoacids, such as valeric acid and this is used as a primary substrate by cellulolytic bacteria. Thus it is believed that if available sugar is low or limiting then the cellulolytic bacteria cannot utilise the cellulose due to a lack of isoacids, and the efficiency of fermentative digestion falls rapidly. However, cellulolytic bacteria in the gut can also obtain energy from keto-acids derived from deamination of amino acids. If the hydrolysis of amino acids and rate of ammonia production is greater than utilization for microbial protein, the ruminal ammonia and plasma urea levels will increase greatly, resulting in a wastage of nitrogen and possible urea toxicosis to the animal (Becht, 1987). While not wishing to be bound by theory, it is believed that the administration of a carbohydrate facilitates keto-acid production by amylose degrading bacteria and so provides an alternative source of keto-acids which is used in preference to keto-acids derived from deamination of amino acids. As a result, amino acids present in the gut will be incorporated directly into protein, and ammonia production will be lower than it otherwise might be.

Administration of a physiologically acceptable composition which comprises a carbohydrate capable of acting as an energy source and/or carbon source for amylose degrading bacteria provides a benefit in excess of the benefit that could be expected based on its calorific content alone. If the carbohydrate is used to replace an energy source such as grain, an amount of the carbohydrate which is less than the equivalent in grain can be used. Thus there is a net energy gain.

Any carbohydrate that can be the energy source and/or carbon source for amylose degrading bacteria is suitable. In an embodiment, the carbohydrate is a mono- or disaccharide.

In an embodiment the carbohydrate is a disaccharide. In an embodiment the carbohydrate is sucrose.

In an embodiment the carbohydrate is a monosaccharide. In an embodiment the carbohydrate is a monosaccharide selected from the group consisting of glucose, fructose and dextrose.

In an embodiment, the carbohydrate is dextrose.

In an embodiment, the carbohydrate comprises 40 to 75% of total agent composition.

The physiologically acceptable composition may be administered to the animal by any suitable method. The components of the composition may be administered may be administered sequentially, simultaneously or concomitantly.

In an embodiment, the physiologically acceptable composition is formulated as a concentrate for application into the water supply of ruminant animals. The concentrate can be administered by adding a measured amount to a source of drinking water such as a drinking trough. Advantageously the concentrate is metered into drinking water as it is dispensed into a source of drinking water. In particular, it may be proportionally dosed through the Nutridose or NutriPro dosing units (Direct Injection Technologies), or any other proportional dosing unit.

### DESCRIPTION OF EMBODIMENTS

### EXAMPLE 1

Formulation 1 was manufactured as a dry concentrate by mixing water soluble salts of the elements listed in Table 1 in a mixer to produce a composition with the following trace element profile shown in Table 1 below:

TABLE 1

| Analysis | Mg/L | Mg/30 ml |
|---|---|---|
| Cobalt | 1,296.75 | 38.90 |
| Copper | 2,500.00 | 75.00 |
| Magnesium | 3,634.20 | 109.00 |
| Manganese | 8,525.00 | 255.80 |
| Zinc | 8,100.00 | 243.00 |
| Selenium | 1,980.00 | 59.40 |
| Iodine | 6239.00 | 18.70 |
| potassium | 293.60 | 8.80 |
| sodium | 1,980.00 | 59.40 |
| Sulfur | 45,753.25 | 1,372.60 |

During manufacture of formulation 1, a carbohydrate in the form of dextrose is added to a final concentration of substantially 66% or 71%. Formulation 1 is suitable for cattle, sheep and goats during times of stress that occur in weaning, yarding, transport, all types of induction and other periods of animal stress.

This formulation can be measured and poured directly into drinking troughs, or proportionally dosed through a proportional dosing unit. It is fed 1 to 3 days before transport at a dosage rate of 30 ml per head for cattle or 10 mls per head for sheep and goats. Using the expected drinking rate of 25 L per head per day, the formulation is easy to administer by calculating the total expected water intake over the 1, 2 or 3 days the stock will be drinking. Once this is calculated, the required amount of formulation is added into the water supply. A dosage rate is shown in Table 2 below.

TABLE 2

| | | | 1 Day | 2 Days | 3 Days |
|---|---|---|---|---|---|
| | | | of dosing | of Dosing | of Dosing |
| | | Total | Dilution | Dilution | Dilution |
| | Head | Water | rate at | Rate | Rate |
| | | | 25 L | 25 L | 25 L |
| Head | of Sheep/ | Drank | water | water | water |
| of Cattle | Goats | per day | per head | per head | per head |
| in Yard | in Yard | (L) | per day | per day | per day |
| 100 | 300 | 2500 | 3.00 L | 1.50 L | 1.00 L |
| 150 | 450 | 3750 | 4.50 L | 2.25 L | 1.50 L |
| 200 | 600 | 5000 | 6.00 L | 3.00 L | 2.00 L |
| 250 | 750 | 6250 | 7.50 L | 3.75 L | 2.50 L |
| 300 | 900 | 7500 | 9.00 L | 4.50 L | 3.00 L |
| 350 | 1050 | 8750 | 10.50 L | 5.25 L | 3.50 L |
| 400 | 1200 | 10000 | 12.00 L | 6.00 L | 4.00 L |
| 450 | 1350 | 11250 | 13.50 L | 6.75 L | 4.50 L |
| 500 | 1500 | 12500 | 15.00 L | 7.50 L | 5.00 L |
| 550 | 1650 | 13750 | 16.50 L | 8.25 L | 5.50 L |

Alternatively, formulation 2 can be dosed very simply with a direct injection system by simply setting the flow trigger to 25 L and the dose rate to reflect either 1 day, 2 days or 3 days that stock have access to the supplemented water. The need to calculate, and premix the formula is eliminated. Dosing is at the rates shown in Table 3:

TABLE 3

| | 1 Day | 2 Days | 3 Days |
|---|---|---|---|
| | of Dosing | of Dosing | of Dosing |
| Water | Direct | Direct | Direct |
| Flow | Injection | Injection | Injection |
| Trigger | Dose | Dose | Dose |
| 25 L | 30 mls | 15 mls | 10 mls |

If a repeat dose is required or desired, it should be given 6 weeks after the last day of dosing and then another 30 ml is administered. Additional nutrient ingredients can be added during manufacture without departing from the scope of the present invention.

The present invention provides a number of advantages over the prior art such as improved ease of use in reducing or treating “Transit Tetany”; “Dead Bellies” in feedlot induction cattle; Bovine Respiratory Disease and assisting in “Sick Pen” recovery; and can reduce reliance on induction treatments.

### EXAMPLE 2

Formulation 2 was prepared as described in Example 1 so that, when given to cattle at the rate of 30 ml per head per day via the drinking water, the amounts of active ingredient provided are as listed in Table 4 below:

TABLE 4

| Analysis | mg/30 ml |
|---|---|
| Cobalt | 7.40 |
| Copper | 210.00 |
| Magnesium | 300.00 |
| Manganese | 602.31 |
| Zinc | 578.56 |
| Selenium | 7.26 |
| Iodine | 18.72 |
| potassium | 8.81 |
| sodium | 7.26 |
| Dextrose | 4500.00 |

This product is a soluble formulation of the active ingredients, dextrose, copper, cobalt, manganese, iodine, selenium, sulfur and magnesium. While the ingredients and their amounts would not be expected to significantly affect growth rates, when fed to cattle a surprising effect on the growth and well-being of cattle is observed.

In a trial at Toowoomba, Australia, conducted over 35 days, cattle fed Formulation 2 gained 200 grams per day, on average, more than the untreated group. They were also observed to be calmer, with less aggressive behaviour.

### EXAMPLE 3

Formulation 3 was prepared by taking 500 litres of a trace element mix comprising potassium iodide, cobalt sulfate, copper sulfate, zinc sulfate, sodium selenite, manganese sulfate and dextrose in the amounts set out in Table 5 with a further 150 kg dextrose and 100 kg magnesium sulfate. The concentration of the components when it is given to cattle variously rates of 10, 30 and 50 ml per head per day via the drinking water is shown in the right hand columns.

TABLE 5

| | | | Active | | | | | | |
|---|---|---|---|---|---|---|---|---|---|
| | kg/ | percent | kg/ | Active | Active | | | | |
| | tonne | active | tonne | in 125 kg | mg/l | mg/ml | mg/10 ml | mg/30 ml | mg/50 ml |
| Potassium | 3.67 | 68 | 2.4956 | 0.31195 | 311.95 | 0.31195 | 3.1195 | 9.3585 | 15.5975 |
| Iodide | | | | | | | | | |
| Cobalt | 4.7 | 21 | 0.987 | 0.123375 | 123.375 | 0.123375 | 1.23375 | 3.70125 | 6.16875 |
| Sulfate | | | | | | | | | |
| Copper | 112 | 25 | 28 | 3.5 | 3500 | 3.5 | 35 | 105 | 175 |
| sulfate | | | | | | | | | |
| Zinc Sulfate | 214.28 | 36 | 77.1408 | 9.6426 | 9642.6 | 9.6426 | 96.426 | 289.278 | 482.13 |
| Sodium | 2.2 | 44 | 0.968 | 0.121 | 121 | 0.121 | 1.21 | 3.63 | 6.05 |
| selenite | | | | | | | | | |
| manganese | 259.06 | 31 | 80.3086 | 10.038575 | 10038.575 | 10.038575 | 100.38575 | 301.15725 | 501.92875 |
| sulfate | | | | | | | | | |
| dextrose | 404.09 | 100 | 404.09 | 50.51125 | 50511.25 | 50.51125 | 505.1125 | 1515.3375 | 2525.5625 |
| | 1000 | | | | | 0 | 0 | 0 | 0 |
| | | | | | | 0 | 0 | 0 | 0 |
| Magnesium | 100 | 9.7 | 9.7 | | 9700 | 9.7 | 97 | 291 | 485 |
| Sulfate | | | | | | | | | |
| Dextrose | 150 | | 150 | | 150000 | 150 | 2005.1125 | 6015.3375 | 7500 |

A typical analysis is shown in Table 6.

TABLE 6

| Analysis | mg/l | g/L | g/ml | grams per 50 ml |
|---|---|---|---|---|
| Cobalt | 123.375 | 0.123375 | 0.000123375 | 0.00616875 |
| Copper | 3500 | 3.5 | 0.0035 | 0.175 |
| Magnesium | 9700 | 9.7 | 0.0097 | 0.485 |
| Manganese | 10038.575 | 10.038575 | 0.01004 | 0.50192875 |
| Zinc | 9642.6 | 9.6426 | 0.0096426 | 0.48213 |
| Selenium | 121 | 0.121 | 0.000121 | 0.00605 |
| Iodine | 311.95 | 0.31195 | 0.00031195 | 0.0155975 |
| Potassium | 293.6 | 0.2936 | 0.0002936 | 0.01468 |
| Sodium | 242 | 0.242 | 0.000242 | 0.0121 |
| Glucose | 200511.25 | 200.51125 | 0.2005113 | 10.0255625 |

Animals were dosed continuously with Formulation 3 while in a holding yard prior to transit for a week and then when in transit for a period of 11 days. The composition was administered through a Nutridose system (Direct Injection Systems Pty Ltd) to proportionally dose the composition throughout the drinking water supply. The Nutridose system is a microprocessor-controlled system that utilises an electronic water meter to measure water flow and trigger the correct dose accordingly. The Nutridose system was modified to work on a 1 L pulse RFPS paddle wheel water meter. This enabled the average 24-hour (per head) drink rate to be used to precisely dose the exact amount. The recommended dose administered was maintained as close as possible to 50 mIs per head per day.

The cattle were split into a treated mob of cattle and a mob of non-treated cattle to provide a control. The mobs comprised a mix of light bulls, heifers, cows, super steers and feedlot steers. Every day from the day of departure right through to the day of discharge, the cattle were monitored and observed twice daily. The cattle in both the treated and untreated decks were observed for the following.
Temperament—Were the cattle calm, agitated, distressed, uncomfortable, aggressive, frightened or depressed?
Condition—Were the cattle putting on weight, displaying energy, displaying a shiny or healthy coat, looking hydrated?
Consumption—Were the cattle consuming more, about the same, or less feed and water?

Whilst observing the cattle in the different mobs, it was observed that the treated cattle were calmer and more likely to be lying down than the non-treated cattle. Calmness was also indicated in the way treated cattle would pull back from the feed troughs, but would return back to the feed trough much more quickly than the untreated cattle. There were no distressed or agitated cattle observed in the treated mob but there were some distressed or agitated cattle observed in the un-treated mob.

Overall, the cattle in the treated mob appeared to be more energetic and fuller. No mortalities occurred in the treated mob. In fact, the hospital pen for the treated mob had cattle from the untreated mob in it, and it was noted that these cattle managed to hold on and not slip further backwards once moved to the hospital pen with treated water.

It was also observed was that the treated cattle did appear to have an increased appetite. During the daily observations it was noted that every single feed trough for the treated mob was completely empty. By comparison, the feed troughs for the untreated mob, whilst still considerably empty, did often have a noticeable amount of feed left in the trough.

When unloaded the treated cattle settled in to the feedlot and were expressing behaviour consistent with freedom four (The Five Freedoms of Animal Welfare) “Freedom to express normal behaviour”. This was evidenced by the treated cattle feeding on the hay provided to them in the feedlot. When these cattle were approached, the temperament remained calm as the cattle continued to eat and did not become “flighty”. In comparison to this, the untreated cattle were found in their pen to be “flighty” and avoiding contact from the observers. They were observed to be not interested in feeding, and when approached by people, took flight as a mob.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

### REFERENCES

The following documents are referred to herein, and their disclosure is incorporated herein by reference:
Becht, Richard R. (1987) “Effects of Isoacids on Ruminal Metabolism and Milk Production,” Iowa State University Veterinarian: Vol. 49: Iss. 1, Article 3.
Available at: https://lib.dr.iastate.edu/iowastate_veterinarian/vol49/iss1/3
Gortel, K., Schaefer, A. L., Young, B. A., and Kawamoto, S. C. (1992)—Effects of transport stress and electrolyte supplementation on body fluids and weights of bulls. Can. J. Anim. Sci. 72: 547-553.
Jones, S. D. M., Schaefer, A. L. and Tong, A. K. W. (1992)—The effects of fasting, electrolyte supplementation and electrical stimulation on carcass yield and meat quality in bulls. Can. J. Anim. Sci. 72: 791-798.
Schaefer, A. L., Jones, S. D. M., Tong, A. K. W. and Young, B. A. (1990)—Effects of transport and electrolyte supplementation on ion concentrations, carcass yield and quality in bulls. Can. J. Anim. Sci. 70: 107-119.
Schaefer, A. L., Jones, S. D. M., Tong, A. K. W., Young, B. A., Murray, N. L. and Lepage, P. (1992)—Effects of posttransport electrolyte supplementation on tissue electrolytes, haematology, urine osmolality and weight loss in beef bulls. Livest. Prod. Sci. 30: 333-346.
Wythes, J. R., Shorthouse, W. R., Schmidt, P. J., and Davis, C. B. (1980)—Effects of Various Rehydration Procedures after a long Journey on Liveweight, Carcasses and Muscle Properties of Cattle. Aust. J Agric. Res. 31:849-855.
Wythes, J. R., Brown, M. J., Shorthouse, W. R., and Clarke, M. R. (1983)—Effect of method of sale and various water regimes at saleyards on the liveweight, carcass traits and muscle properties of cattle. Aust. J. exp. Agric. Anim. Husb. 23:235-242.

1. A composition for reducing transport stress in a ruminant animal which is suitable for administration in the drinking water of the ruminant animal, comprising:
(a) a carbohydrate capable of acting as an energy source and/or carbon source for amylose degrading bacteria;
(b) a source of magnesium; and
(c) trace elements comprising:
a source of iodine;
a source of cobalt;
a source of copper;
a source of zinc;
a source of selenium; and/or
a source of manganese.

2. The composition as claimed in claim 1, wherein the source of iodine is substantially 0.01 to 0.50% of total agent composition.

3. (canceled)

4. The composition as claimed in claim 1, wherein the source of cobalt is substantially 0.01 to 1.00% of total agent composition.

4. The composition as claimed in claim 1, wherein the source of copper is substantially 0.01 to 1.00% of total agent composition.

5. (canceled)

6. (canceled)

7. (canceled)

8. The composition as claimed in claim 1, wherein the source of zinc is substantially 0.50 to 2.00% of total agent composition.

9. (canceled)

10. The composition as claimed in claim 1, wherein the source of selenium is substantially 0.01 to 0.50% of total agent composition.

11. (canceled)

12. The composition as claimed in claim 1, wherein the source of manganese is substantially 0.50 to 2.50% of total agent composition.

13. (canceled)

14. The composition as claimed in any one of claims 1 to 13, wherein the source of magnesium is substantially 0.1 to 10% of total agent composition.

15. (canceled)

16. The composition as claimed in claim 1, wherein the carbohydrate is a mono- or disaccharide.

17. The composition as claimed in claim 16, wherein the carbohydrate is a monosaccharide selected from the group consisting of glucose, fructose and dextrose.

18. The composition as claimed in claim 17, wherein the carbohydrate is dextrose.

19. The composition as claimed in claim 1, wherein the carbohydrate comprises 40 to 75% of total agent composition.

20. The composition as claimed in claim 1, wherein a source of sulfur is also administered.

21. The composition as claimed in claim 20, wherein the source of sulfur is substantially 5% of total agent composition.

22. A method of reducing transport stress in a ruminant animal, comprising administering:
(a) a carbohydrate capable of acting as an energy source and/or carbon source for amylose degrading bacteria;
(b) a source of magnesium; and
(c) trace elements comprising:
a source of iodine;
a source of cobalt;
a source of copper;
a source of zinc;
a source of selenium; and/or
a source of manganese;
in the drinking water of the ruminant animal.

23. The method as claimed in claim 22, wherein cellulose digestion is increased.

24. The method as claimed in claim 22, wherein net available energy is increased.

25. The method as claimed in claim 22, wherein physiological stress is reduced.

26. A kit comprising:
(a) a carbohydrate capable of acting as an energy source and/or carbon source for amylose degrading bacteria;
(b) a source of magnesium;
(c) trace elements comprising:
a source of iodine;
a source of cobalt;
a source of copper;
a source of zinc;
a source of selenium; and/or
a source of manganese; and
(d) instructions for administering the carbohydrate, source of magnesium and trace elements to the drinking water of a ruminant animal.

27. A kit as claimed in claim 26, further comprising a source of sulfur, and wherein the instructions further comprise instructions for administering the source of sulfur to the drinking water of a ruminant animal.
